(19) United States
(12) Patent Application Publication
(10) Pub. No.: US 2012/0190890 A1
(43) Pub. Date: Jul. 26, 2012

(54) METHOD FOR PRODUCING BIORESOURCED ACRYLIC ACID FROM GLYCEROL

(75) Inventor: Michel Fauconet, Valmont (FR)

(73) Assignee: ARKEMA FRANCE, Colombes (FR)

(21) Appl. No.: 13/386,096

(22) PCT Filed: Jun. 29, 2010
(86) PCT No.: PCT/FR2010/051363

§ 371 Date: Apr. 6, 2012

(30) Foreign Application Priority Data
Jul. 22, 2009 (FR) 0955112

(51) Int. Cl. C07C 51/16

(52) U.S. Cl. 562/538

(57) ABSTRACT

An aim of the present invention is to produce, from glycerol, a bioresourced acrylic acid, that is to say an acrylic acid essentially based on a carbon source of natural origin, corresponding to a degree of purity corresponding to the requirements of the users.

The process according to the invention comprises a final stage of purification of the acrylic acid by fractional crystallization applied to one of the acrylic acid fractions resulting from the purification line employed after having extracted the acrylic acid, obtained from glycerol, by countercurrentwise absorption with a heavy hydrophobic solvent.

The present invention is targeted at a process for the manufacture of a bioresourced acrylic acid from glycerol as starting material, the term “bioresourced acid” indicating that the acrylic acid is essentially based on a carbon source of natural origin.

Acrylic acid is a very important starting material which can be used directly to produce an acrylic acid polymer or, after esterification with alcohols, to produce a polymer of the corresponding ester. These polymers are used as is or as copolymers in fields as varied as hygiene (for example, in the production of superabsorbants), detergents, paints, varnishes, adhesives, paper, textiles, leather, and the like.

Manufacturers have been developing processes for the synthesis of acrylic acid for decades.

A first generation used, as starting material, compounds comprising a triple bond of acetylenic type which were reacted with a mixture of carbon monoxide and water in the presence of a nickel-based catalyst.

The second generation of processes, which is today the main process for the production of acrylic acid, is based on the oxidation of propylene and/or propane. These starting materials result from oil and consequently the acrylic acid is formed from a nonrenewable fossil carbon-based starting material. In addition, the processes for extracting, purifying and synthesizing the starting materials and the processes for destroying, at the end of the cycle, the manufactured finished products based on these fossil starting materials generate carbon dioxide, the latter being a direct byproduct of the reactions for the oxidation of propylene to give acrolein and then of acrolein to give acrylic acid. All this contributes to increasing the concentration of greenhouse gases in the atmosphere. In the context of the commitments of the majority of industrialized countries to reduce emissions of greenhouse gases, it appears particularly important to manufacture novel products based on a renewable starting material, contributing to reducing these environmental effects.

For several years, manufacturers have directed their research and development studies at “bioresourced” synthetic processes using naturally renewable starting materials. Specifically, in order to limit the ecological impact of conventional production processes, alternative processes starting from nonfossil plant starting materials have recently been developed. Examples are processes using, as starting material, 2-hydroxypropionic acid (lactic acid) obtained by fermentation of glucose or molasses originating from the biomass. Further processes are those starting from glycerol (also known as glycerin), resulting from the methanolysis of vegetable oils at the same time as the methyl esters, which are themselves employed in particular as fuels in gas oil and domestic heating oil. This glycerol is a natural product which enjoys a “green” aura; it is available in large amounts and can be stored and transported without difficulty. The methanolysis of vegetable oils or animal fats can be carried out according to various well-known processes, in particular by using homogeneous catalysis, such as sodium hydroxide or sodium methoxide in solution in methanol, or by using heterogeneous catalysis. Reference may be made on this subject to the paper by D. Ballerini et al. in l'Actuante Chimique of November-December 2002.

The processes using hydroxypropionic acid as starting material have a major disadvantage from the economic viewpoint. They involve a fermentation reaction which is necessarily carried out under highly dilute conditions in water. In order to obtain acrylic acid, a very large amount of water has to be removed by distillation, at the price of a very high energy cost. Furthermore, the energy expended to separate the water, which energy is produced from fossil material, will be highly damaging to the initial advantage of producing acrylic acid from this bioresourced starting material. Mention may be made, in this field, of application WO2006/092271, which describes a process for the production of polymers from acrylic acid prepared by the enzymatic route, in particular from carbohydrate.

As regards the conversion of glycerol by the chemical route, mention may be made of the two-stage synthesis of acrylic acid, namely the production of acrolein by dehydration of glycerol, which is described in particular in the U.S. Pat. No. 5,387,720, followed by a “conventional” oxidation of the acrolein to produce the acrylic acid.

The first stage of the manufacture of acrylic acid from glycerol results in the same intermediate compound as the conventional manufacturing process starting from propylene, namely acrolein, according to the reaction:

CH₂OH—CHOH—CH₂OH→CH₂═CH—CHO+2H₂O

which is followed by the second stage of oxidation, according to the reaction:

CH₂═CH—CHO+½O₂→CH₂═CH—COOH

Patent applications EP 1 710 227, WO2006/136336 and WO2006/092272 describe such processes for the synthesis of acrylic acid from glycerol comprising the stage of gas-phase dehydration in the presence of catalysts composed of inorganic oxides (which may or may not be mixed) based on aluminum, titanium, zirconium, vanadium, and the like, and the stage of gas-phase oxidation of the acrolein thus synthesized in the presence of catalysts based on oxides of iron, molybdenum or copper, alone or in combination in the form of mixed oxides.

Acrylic acid is intended for the use by manufacturers of processes for the polymerization either of acrylic acid or of its ester derivatives, which processes are carried out under various forms, in bulk, in solution, in suspension or in emulsion. These processes can be highly sensitive to the presence in the charge of certain impurities, such as aldehydes or unsaturated compounds, which can sometimes prevent the expected use value from being obtained, for example by limiting the conversion of the monomer to give the polymer, by limiting the chain length of the polymer or by interfering in the polymerization in the case of unsaturated compounds. Other impurities, such as nonpolymerizable saturated compounds, can be particularly troublesome in the final application by modifying the properties of the finished product, by conferring toxic or corrosive properties on the finished product or by increasing polluting organic discharges during the stages of manufacture of the polymer and/or of the finished product.

Operators are proving to be demanding as regards quality specifications for acrylic acid (or for its ester). The latter must meet strict thresholds as regards impurities. Specifically, users of acrylic acid or of acrylic esters which produce polymers employ formulations suited to the production of their polymers from a “standard” grade of acrylic acid or of esters today manufactured solely from propylene. A modification to the formulations used by these users, for the purpose of adapting them to a different grade of acrylic acid or of esters produced by a route other than that of the conventional ex-propylene processes, would exhibit significant disadvantages for these user companies. Apart from the additional research and development costs, the production of one type of polymer on the same unit starting from different grades of acrylic acid or of esters according to their origin, fossil or bioresourced (such as glycerol), would occasion significant conversion costs and a more complicated production infrastructure. As the grade of the acrylic acid, that is to say its content of various impurities, plays a major role in the subsequent polymerization processes, the manufacturers manufacturing this acrylic acid have been led to deploy a whole series of purification stages in order to obtain this “standard” acrylic acid which is normally referred to as glacial acrylic acid (GAA). This GAA does not correspond to specifications officially to recognized and having a universal nature but means, for each manufacturer, the level of purity to be achieved in order to be able to successfully carry out his subsequent conversions. By way of example, for an ex-propylene acrylic acid, the reactor outlet effluent stream is subjected to a combination of stages which can differ in their sequence according to the process: removal of the noncondensable compounds and most of the very light compounds, in particular the intermediate acrolein for the synthesis of the acrylic acid (crude AA), dehydration removing the water and the formaldehyde (dehydrated AA), removal of the light compounds (in particular acetic acid), removal of the heavy compounds, and optionally removal of some residual impurities by chemical treatment.

The invention is targeted at a process for the manufacture of a “standard” acrylic acid by using glycerol as starting material which will be converted in two stages—dehydration and oxidation—as mentioned above, incorporated in an overall purification process.

This process is highly analogous to the synthesis process starting from propylene insofar as the intermediate product, acrolein, resulting from the first stage is the same and in that the second stage is carried out under the same operating conditions. However, the reaction of the first stage of the process of the invention, the dehydration reaction, is different from the reaction for the oxidation of propylene of the normal process. The dehydration reaction, performed in the gas phase, is carried out using solid catalysts different from those used for the oxidation of propylene. The acrolein-rich effluent stream resulting from the first dehydration stage, intended to feed the second stage of oxidation of the acrolein to give acrylic acid, comprises a greater amount of water and additionally exhibits substantial differences as regards byproducts resulting from the reaction mechanisms involved being given material form by different selectivities in each of the two routes.

In order to illustrate these differences, the data relating to the presence of various acids in the crude acrylic acid, that is to say in the liquid phase exiting from the second-stage reactor, are collated in the following table 1.

TABLE 1

| Impurity/AA ratio by weight | Ex-propylene | Ex-glycerol |
|---|---|---|
| (crude acrylic acid) | process | process |
| Acetic acid/AA | <5% | >10% |
| Propionic acid/AA | <0.1% | >0.5% |
| 2-Butenoic acid/AA | <0.001% | >0.01% |

The impurities/AA ratios depend on the catalysts used, on their “age” (deterioration in the selectivities over time) and on the operating conditions. In table 1, the 2-butenoic acid/AA ratio is given as <0.001% for the ex-propylene process; however, although the Applicant Company has never detected it in ex-propylene AA, it considers it preferable to write “<10 ppm” rather than 0% (result of its analysis) in order to eliminate the problem of detection threshold related to the analytical method.

Some of the main differences, in terms of constituents of the liquid effluent stream exiting from the oxidation reactor, between the ex-propylene and ex-glycerol processes are illustrated in table 1. Naturally, although this is not mentioned in the table, there is also found, in the crude acrylic acid, whether it originates from the ex-propylene process or from the ex-glycerol process, a whole series of oxygen-comprising compounds, alcohols, aldehydes, ketones, other acids, and the like, the necessary separation of which is known to a person skilled in the art.

The specifications for the acrylic acid grades commonly used for the production of acrylic acid and acrylic ester polymers require reducing the contents of the impurities of table 1 in acrylic acid down to the values which appear in table 2 below.

TABLE 2

| Concentration of | Technical | Glacial acrylic |
|---|---|---|
| the impurities in | acrylic acid for | acid for |
| the AA (by weight) | esterification | polymerization |
| Acetic acid | <0.2% | <0.1% |
| Propionic acid | <0.05% | <0.05% |
| 2-Butenoic acid | <0.005% | <0.001% |

The acetic acid and the propionic acid are troublesome in particular because they are not converted during the polymerization process; they are saturated and thus cannot be polymerized. According to the polymerization process involved and the applications targeted for the polymer, these impurities may remain in the finished product and risk conferring undesirable corrosive properties on the finished product or may be found in the liquid or gaseous discharges generated by the polymerization process and cause equally undesirable organic pollution.

The 2-butenoic acid, not synthesized by the ex-propylene process but present in both its configurations (E, also known as crotonic acid, CAS No.: 107-93-7, and Z, also known as isocrotonic acid, CAS No.: 503-64-0) in the ex-glycerol process, is for its part particularly troublesome because, due to its double bond, it is capable of participating in the polymerization process and thus of modifying the characteristics and the use value of the final polymer.

The problem posed is that of producing an acrylic acid with a degree of purity corresponding to the requirements of the users and meeting in particular the specifications given in table 2 on carrying out a process for the synthesis of acrylic acid using glycerol as starting material which exhibits the disadvantage, compared with the conventional process for the oxidation of propylene, of providing, at the outlet of the oxidation reactor, a gas mixture comprising a great deal of water and exhibiting high contents of various impurities, such as acetic acid, propionic acid and 2-butenoic acid. The Applicant Company has discovered that it is possible to overcome the preceding disadvantages by employing a process for the purification of the gaseous effluent stream resulting from the oxidation reactor of a process for the synthesis of acrylic acid from glycerol, comprising a first stage of dehydration of the glycerol followed by a second stage of oxidation of acrolein, combining a stage of absorption of the acrylic acid by a heavy solvent at the outlet of the oxidation reactor and a multistage purification phase ending with a separation of the acrylic acid by fractional crystallization.

The employment of a stage of absorption of the acrylic acid by a heavy solvent makes it possible to solve, upstream, most of the problems posed by the presence of water and light impurities soluble in the aqueous phase, and the purification by fractional crystallization makes it possible to remove, all at the same time, the traces of heavy impurities, of intermediate impurities, that is to say those having a boiling point between that of acrylic acid and that of the heavy solvent, essentially composed of “heavy” compounds originating from the dehydration and oxidation reactions and optionally some polymerization-inhibiting stabilizers, and remaining light impurities and in particular propionic acid.

A subject matter of the invention is a process for the manufacture of bioresourced acrylic acid from glycerol, comprising the following stages:
gas-phase catalytic dehydration of glycerol to give acrolein, (1)
partial condensation by cooling and extraction of a portion of the water and heavy compounds present in the reaction medium of (1), (1′)
gas-phase catalytic oxidation of the acrolein to give acrylic acid, (2)
extraction of the acrylic acid present in the effluent stream from the oxidation by countercurrentwise absorption with a heavy hydrophobic solvent with cooling and removal, at the top, of the light fraction composed of the “noncondensable” gaseous compounds and condensable light compounds, such as water, acetaldehyde, unconverted acrolein, formic acid or acetic acid, (3)
separation of the residual light fraction and the heavy solvent present in the liquid phase resulting from stage (3) by at least one distillation stage (4), (5) and/or (6) and recovery of the acrylic acid fraction thus separated, and
purification of the acrylic acid present in the acrylic acid fraction resulting from the preceding stage(s) by fractional crystallization.

In a first preferred alternative form of the process of the invention, the liquid phase resulting from stage (3) is subjected to
a topping by distillation with separation, at the top, of water and residual light compounds (stage 4), the bottom fraction being sent to stage (5),
a distillation of the acrylic acid solution thus obtained in order to separate, at the bottom, the heavy solvent and, at the top, the acrylic acid fraction comprising the intermediate impurities and optionally the traces of solvent (stage 5),
a distillation of the acrylic acid solution resulting from the preceding stage (5) in order to remove, at the bottom, the heaviest “intermediate” compounds and the traces of solvents possibly entrained and, at the top, the acrylic acid (stage 6),
a purification of the acrylic acid resulting from stage (6) by fractional crystallization.

Glycerol is a chemical, 1,2,3-propanetriol, which can be obtained either by chemical synthesis, starting from propylene, or as coproduct formed during the methanolysis of vegetable oils or animal fats.

The methanolysis of vegetable oils or animal fats can be carried out according to various well-known processes, in particular by using homogeneous catalysis, such as sodium hydroxide or sodium methoxide in solution in methanol, or by using heterogeneous catalysis. Reference may be made on this subject to the paper by D. Ballerini et al. in l'Actualite Chimique of November-December 2002.

The methanolysis of vegetable oils results, on the one hand, in methyl esters and, on the other hand, in glycerol. The methyl esters are employed in particular as fuels in gas oil and domestic heating oil. With the development of fuels having renewable origins, in particular vegetable oil methyl esters (VOMEs), the production of glycerol according to this production route has greatly increased, the glycerol representing of the order of 10% of the weight of the oil converted.

The glycerin, the name of glycerol when it is in aqueous solution, obtained from vegetable oils or animal fats can comprise salts (NaCl, Na₂SO₄, KCl, K₂SO₄). In this case, a preliminary stage of removal of the salts, for example by distillation, by use of ion-exchange resins or by use of a fluidized bed, such as described in French application FR 2 913 974, will generally be present. Mention will in particular be made, among the methods used or studied for the purification and the evaporation of glycerol, of those which are described by G. B. D'Souza in J. Am. Oil Chemists' Soc., November 1979 (Vol 56), 812A, by Steinberner U. et al. in Fat. Sci. Technol. (1987), 89, Jahrgang No. 8, pp 297-303, and by Anderson D. D. et al. in Soaps and Detergents: A Theoretical and Practical Review, Miami Beach, Fla., Oct. 12-14, 1994, chapter 6, pp 172-206. Ed: L Spitz, AOCS Press, Champaign.

Use is generally made, for the implementation of the process, of a stream feeding the reactor of stage (1) comprising glycerol and water with a water/glycerol ratio by weight which can vary within wide limits, for example between 0.04/1 and 9/1 and preferably between 0.7/1 and 3/1.

The principle of the process for obtaining acrylic acid from glycerol is based on the 2 consecutive dehydration and oxidation reactions:

CH₂OH—CHOH—CH₂OH [symbol] CH₂═CH—CHO+2H₂O

CH₂═CH—CHO+½O₂→CH₂═CH—COOH

The process is carried out in two separate stages with two different catalysts.

The dehydration reaction, stage (1), which is an equilibrium reaction but one promoted by a high temperature level, is generally carried out in the gas phase in the reactor in the presence of a catalyst at a temperature ranging from 150° C. to 500° C., preferably between 250° C. and 350° C., and an absolute pressure between 1 and 5 bar (100 and 500 kPa). It can also be carried out in the liquid phase. It can also be carried out in the presence of oxygen or of an oxygen-comprising gas, as described in applications WO 06/087083 and WO 06/114506.

The glycerol dehydration reaction is generally carried out over solid acid catalysts. The catalysts which are suitable are substances used in a gaseous or liquid reaction medium, in the heterogeneous phase, which have a Hammett acidity, denoted H₀, of less than +2. As indicated in U.S. Pat. No. 5,387,720, which refers to the paper by K. Tanabe et al. in “Studies in Surface Science and Catalysis”, Vol. 51, 1989, chap. 1 and 2, the Hammett acidity is determined by amine titration using indicators or by adsorption of a base in the gas phase.

These catalysts can be chosen from natural or synthetic siliceous substances or acidic zeolites; inorganic supports, such as oxides, covered with mono-, di-, tri- or polyacidic inorganic acids; oxides or mixed oxides or heteropolyacids or heteropolyacid salts.

These catalysts can generally be composed of a heteropolyacid salt in which the protons of said heteropolyacid are exchanged with at least one cation chosen from elements belonging to Groups I to XVI of the Periodic Table of the Elements, these heteropolyacid salts comprising at least one element chosen from the group consisting of W, Mo and V.

Mention may particularly be made, among mixed oxides, of those based on iron and on phosphorus and of those based on cesium, phosphorus and tungsten.

The catalysts are chosen in particular from zeolites, Nafion® composites (based on sulfonic acid of fluoropolymers), chlorinated aluminas, phosphotungstic and/or silicotungstic acids and acid salts, and various solids of the type comprising metal oxides, such as tantalum oxide Ta₂O₅, niobium oxide Nb₂O₅, alumina Al₂O₃, titanium oxide TiO₂, zirconia ZrO₂, tin oxide SnO₂, silica SiO₂ or silicoaluminate SiO₂/Al₂O₃, impregnated with acid functional groups, such as borate BO₃, sulfate SO₄, tungstate WO₃, phosphate PO₄, silicate SiO₂ or molybdate MoO₃ functional groups, or a mixture of these compounds.

The preceding catalysts can additionally comprise a promoter, such as Au, Ag, Cu, Pt, Rh, Pd, Ru, Sm, Ce, Yt, Sc, La, Zn, Mg, Fe, Co, Ni or montmorillonite.

The preferred catalysts are phosphated zirconias, tungstated zirconias, silica zirconias, titanium or tin oxides impregnated with tungstate or phosphotungstate, phosphated aluminas or silicas, heteropolyacids or heteropolyacid salts, iron phosphates and iron phosphates comprising a promoter.

The reaction medium exiting from the dehydration reactor has a high water content due to the glycerol charge (aqueous solution) and the reaction itself. An additional stage (1′) of partial condensation of the water, such as, for example, that described in patent application WO 08/087,315 on behalf of the Applicant, will make it possible to remove a portion thereof, so as to bring this gas to a composition substantially identical to that of the ex-propylene process, in order to feed the second stage of oxidation of the acrolein to give acrylic acid. The term “composition substantially identical” is understood to mean in particular similar acrolein, water and oxygen concentrations. This condensation stage (1′) has to be carried out with cooling to a temperature which makes it possible to obtain, after removal of the condensed phase, a gas stream comprising water and acrolein in a water/acrolein molar ratio of 1.5/1 to 7/1. This partial condensation of the water makes it possible to prevent damage to the catalyst of the 2^{nd} stage of oxidation of the acrolein to give acrylic acid and to prevent, during the subsequent stages, the removal of large amounts of water, the subsequent removal of which is expensive and which presents the risk of resulting in losses of acrylic acid. In addition, it makes it possible to remove a portion of the “heavy” impurities formed during the dehydration.

The oxidation reaction, stage (2), is carried out in the presence of molecular oxygen or of a mixture comprising molecular oxygen, at a temperature ranging from 200° C. to 350° C., preferably from 250° C. to 320° C., and under a pressure ranging from 1 to 5 bar, in the presence of an oxidation catalyst.

Use is made, as oxidation catalyst, of any type of catalyst well-known to a person skilled in the art for this reaction. Use is generally made of solids comprising at least one element chosen from the list Mo, V, W, Re, Cr, Mn, Fe, Co, Ni, Cu, Zn, Sn, Te, Sb, Bi, Pt, Pd, Ru and Rh, present in the metallic form or in the oxide, sulfate or phosphate form. Use is made in particular of the formulations comprising Mo and/or V and/or W and/or Cu and/or Sb and/or Fe as main constituents.

The gas mixture resulting from stage (2) is composed, apart from acrylic acid:
of light compounds which are noncondensable under the temperature and pressure conditions normally employed: nitrogen, unconverted oxygen, carbon monoxide and carbon dioxide, which are formed in a small amount by final oxidation,
of condensable light compounds: in particular water, generated by the dehydration reaction or present as diluent, unconverted acrolein, light aldehydes, such as formaldehyde and acetaldehyde, formic acid, acetic acid and propionic acid,
of heavy compounds: furfuraldehyde, benzaldehyde, maleic acid, maleic anhydride, 2-butenoic acid, benzoic acid, phenol and protoanemonin.

The gaseous effluent stream resulting from stage (2) is subjected to a stage (3) of countercurrentwise absorption using a heavy hydrophobic solvent which is accompanied by a cooling of the assembly. The gaseous effluent stream is introduced at the bottom of a column and the heavy solvent is introduced at the column top. The flow rate of solvent introduced at the column top is from 3 to 6 times by weight that of the acrylic acid in the gaseous feed mixture. A heavy solvent solution is collected at the column bottom having an acrylic acid content generally of between 15 and 25% by weight and additionally comprising “intermediate” compounds having a boiling point between that of the heavy solvent and that of the acrylic acid. These intermediate compounds are composed of the heavy products of the reaction: furfuraldehyde, benzaldehyde, maleic acid, maleic anhydride, 2-butenoic acid, benzoic acid, phenol or protoanemonin, and of the stabilizing products introduced into the medium in order to inhibit the polymerization reactions.

The light fraction, exiting at the top, is composed of the light compounds which are noncondensable under the temperature and pressure conditions normally employed: nitrogen, unconverted oxygen, carbon monoxide and carbon dioxide, which are formed in a small amount by final oxidation, and of condensable light compounds: in particular water, generated by the dehydration reaction or present as diluent, unconverted acrolein, light aldehydes, such as formaldehyde and acetaldehyde, formic acid and acetic acid.

This operation of extraction by the heavy hydrophobic solvent is well known and has even been described for the treatment of acrylic acid synthesized by oxidation of propylene; mention may be made, on this subject, of the following patents: French patent No. 1 588 432, French patent No. 2 146 386, German patent No. 4 308 087, European patent No. 0 706 986 and French patent No. 2 756 280, which describe such solvents. These solvents have a boiling point of greater than 170° C., for example of between 200 and 380° C. and preferably between 270 and 320° C. French patent No. 1 588 432 describes the use of aliphatic or aromatic acid esters having a high boiling point. They are generally composed of binary mixtures capable of forming eutectics, such as, for example, diphenyl (DP) and diphenyl ether (DPO), which form a eutectic in the 26.5-76.5 proportions (FP No. 2 146 386 and EP 0 706 986), or even ternary mixtures, DP/DPO/dimethyl phthalate (DMP) (DE No. 4 308 087). French patent No. 2 756 280 recommends the use of aromatic solvents exhibiting a boiling point of greater than 260° C. and comprising one or two aromatic ring systems substituted by at least one alkyl radical having from 1 to 4 carbon atoms or one cycloalkyl radical, in particular ditolyl ether, alone or in the form of a mixture of its isomers, or the ditolyl ether (DTE) and dimethyl phthalate mixture.

The process of the invention can be carried out with these different solvents. However, the preferred solvents are those described in this French patent No. 2 756 280, which, apart from the fact that they improve the separation from the impurities present in the reaction mixture, reduce the phenomenon of entrainment of traces of solvent in the stream of noncondensable compounds recycled to the reaction section and make possible efficient recovery of the polymerization inhibitors.

According to the preferred alternative form of the process of the invention, the liquid solution of acrylic acid in the heavy solvent is subsequently sent to a topping region, stage (4), in order to remove, at the top, the traces of water and light condensable compounds which remain at the bottom of the preceding absorption region. This topping region is fed at the top with the bottom stream from the absorption region. The top stream, enriched in light compounds, is returned to the absorption region for the purpose of removing these light compounds in its top stream.

The liquid solution of topped acrylic acid obtained at the bottom of this region is subsequently sent to the distillation region for the separation of the heavy solvent and the acrylic acid (stage 5); the heavy solvent is extracted at the bottom of said region in order to be recycled, after treatment, in the first stage. The acrylic acid solution comprising most of the intermediate compounds exits at the top of said region. This stream can optionally also comprise a few traces of solvent.

The acrylic acid solution is subsequently sent to a region for separation, on the one hand, of the intermediate compounds, and, on the other hand, of the purified acrylic acid (technical acrylic acid) (stage 6). The intermediate compounds are extracted at the bottom of the region and the technical acrylic acid is extracted at the top of said region.

The technical acrylic acid produced is subsequently sent to the fractional crystallization region.

The various stages of separation by absorption or distillation require, due to the thermodynamic conditions employed, the addition to the treated streams of polymerization inhibitors in order to prevent the formation of heavy compounds prejudicial to the satisfactory operation of the assembly. The polymerization inhibitors generally used for the stages for the purification of the acrylic acid are phenolic products, such as hydroquinone or hydroquinone methyl ether, phenothiazine derivatives, compounds of the family of the thiocarbamates, such as copper di(n-butyl)dithiocarbamate, amino derivatives, such as hydroxylamines, hydroxydiphenylamine or derivatives of the family of the phenylenediamines, nitroxide derivatives of 4-hydroxy 2,2,6,6-tetramethylpiperidine-1-oxyl (TEMPO), such as 4-hydroxy-TEMPO or 4-oxo-TEMPO, or metal salts, such as manganese acetate. These inhibitors can be used alone or in combination and are in addition preferably introduced in combination with an oxygen-comprising gas.

These polymerization inhibitors are generally heavy compounds, the volatility of which is lower than that of acrylic acid, but can in some cases be lighter than the solvent. They are removed at the bottom of the columns, when inhibitors heavier than the solvent are concerned, or are divided between the top stream and the bottom stream for the inhibitors which are lighter or close to the solvent. In the majority of the columns, their concentration in the vapor phase inside the distillation columns is low and insufficient to prevent the initiation of polymers. In order to prevent the appearance and the accumulation of polymers, these additives are usually introduced into the liquid streams feeding the devices, but also at the top and at various points of the columns and devices, so as to provide continuous and homogeneous reflux of solution rich in polymerization inhibitors over all the parts of the devices. Generally, they are conveyed in solution in a liquid, for example in acrylic acid or in the solvent, if the purification stage relates to streams comprising the solvent.

In the process of the invention, the final stage of the procedure for the purification of the bioresourced acrylic acid is a separation by fractional crystallization thus combined with the preceding purification stages.

Fractional crystallization is a well-known separation technique. It can be carried out in various forms, dynamic crystallization, static crystallization or suspension crystallization. Mention may be made, on this subject, of French patent 77 04510 of Feb. 17, 1977 (BASF) and U.S. Pat. No. 5,504,247 (Sulzer) and U.S. Pat. No. 5 831 124 (BASF) and U.S. Pat. No. 6 482 981 (Nippon Shokubai), some of which are targeted at the purification of acrylic acid synthesized by the oxidation of propylene.

The most widely used technique is falling film fractional crystallization, dynamic crystallization, optionally combined with molten medium static crystallization.

Falling film crystallization is generally carried out in a tubular exchanger, in practice multitubular, each tube being fed continuously (at the top) with:
a liquid stream (solution or melt) of the compound to be purified, acrylic acid (AA) in the process, falling as a film, preferably along the internal wall of the tube, received at the tube bottom and recycled at the top (closed loop) for the time necessary for the crystallization of the amount of compound (AA) decided upon by the operator,
a stream of heat-exchange fluid, for example ethylene glycol/water or methanol/water, falling as a film, preferably along the external wall of the tube, also recirculated throughout the crystallization within the tube and which will introduce the cold or the heat necessary for the operation of the stages of each of the steps.

The process is a combination of successive steps, which each comprise 3 stages:
crystallization: the temperature of the heat-exchange fluid is lowered according to a negative temperature gradient from a temperature slightly greater than the crystallization temperature of the acrylic acid in the medium, of the order of 14° C. Crystals are formed as an increasingly thick layer at the surface of the tubes. When approximately from 30 to 80% of AA circulated has crystallized, after draining, the remaining liquid fraction (mother liquors rich in impurities) is transferred into a receiver.
sweating: the temperature of the heat-exchange fluid is increased according to a positive temperature gradient in order to remove, by melting, the impurities trapped in the form of inclusions in the layer of acrylic acid crystals being formed; these are mainly located in the outermost layer i.e. which was in contact with a recirculated stream increasingly rich in impurities. During the sweating, the first molecules to melt are eutectic mixtures of impurities and of AA, the impurities located in the layer of crystals migrate towards the outer layer that which was in contact with the recirculated stream. A small portion of this layer of crystals is thus melted and transferred into a receiver, preferably the same receiver as that for the mother liquors recovered during the crystallization stage. This sweating stage can be replaced by a washing technique, which consists in removing the impurities present at the surface by washing with pure AA, preferably introduced at a temperature slightly greater than the melting point of the layer of AA. However, this technique is a priori less effective.
melting: the temperature of the heat-exchange fluid is rapidly increased above the melting point of AA (14° C.) and should preferably remain below a maximum temperature above which polymerization (explosive) of the medium may be feared: this maximum temperature is of the order of 35-40° C. in order to remain safe in melting the layer of crystals of purified AA. The purified liquid recovered is placed in a second receiver.

Starting from the stream to be purified, the combination of the three stages described represents a first purification step. The purified liquid resulting from this first step can again be subjected to a sequence of the three stages described in a 2^{nd} purification step (purification phase). The mother liquors resulting from this 2^{nd} step are purer than those from the preceding step and can thus be used as a mixture with a new charge of AA to be purified in step No. 1. The same operation can be carried out in a third purification step, it being possible for the mother liquors from this third step to be recycled in the charge of the 2^{nd} step, the pure product being recovered by melting the crystals. Generally, the mother liquors from the “n” purification step can be recycled by mixing them with the feed stream for the “n−1” purification step.

During the purification phases, the polymerization inhibitors present in the mixtures to be purified are treated like impurities and are thus removed in the mother liquors. In order to prevent the formation of polymers in the molten crystallisate, an inhibitor compatible in nature and concentration with the final use of the monomer is preferably added. This addition will in particular be carried out during the final melting stage of a step fed with a stream devoid of polymerization inhibitor, such as, for example, the final “n” purification step fed solely with a purified stream from the “n−1” step.

The mother liquors collected subsequent to the first purification step can be treated in a “−1” step according to the same three-stage process. The crystallisate recovered can be used as supplement for the feed charge of the first step. The mother liquors from the “−1” step are then treated according to the same process for a new separation, the crystallisate of which will participate as charge for the immediately greater step and the mother liquors of which are again subjected to the process in a lower “−2” step. The “−1”, “−2”, and the like, steps constitute the concentration steps (the successive steps make it possible to concentrate the impurities in the mother liquor streams). Generally, the mother liquors from the “n” concentration steps are treated according to the same three-stage process in the subsequent “n−1” step.

The repetition of these operations (concentration phase) will make it possible to concentrate the impurities in a mother liquor stream increasingly rich in impurities, while the pure acrylic acid fractions will be returned to the initial step. Thus, the acrylic acid entrained in the initial mother liquors can be recovered in order to improve the recovery yield and, furthermore, a mixture “enriched” in impurities can be obtained.

The successive concentration steps are characterized by mother liquor streams which are increasingly concentrated in impurities as these steps pile up. In doing this, the crystallization temperature of these mixtures becomes increasingly low, which has the effect of increasing the energy cost of the cooling. Furthermore, the time necessary to crystallize the same amount of acrylic acid becomes increasingly lengthy, which has the consequence of reducing the productive output of the purification for the same crystallization surface area. Consequently, the number of the concentration steps will preferably generally be halted before the total concentration of impurities in the mother liquors exceeds 50% by weight of the stream.

Depending on the purity of the starting material, the purity of the expected purified product and the AA recovery yield desired, the complete process for an initial AA grade of “technical” type comprises at least 2 purification steps, preferably between 2 and 4 purification steps, and between 1 and 4 steps for the concentration of the impurities.

In order to further improve the recovery yield, it is also possible to add a final step of recovery in a static crystallizer. In this case, the mixture to be crystallized is placed in contact with a cold wall. It can, for example, be an exchanger composed of metal sheets, through which a heat-exchange fluid passes, immersed in a vessel comprising the crystallization mother liquors from the preceding steps. The AA forms a crystal layer on the wall of the sheets, the mother liquors are then removed and the crystallized layer is melted in order to be subsequently treated in a higher step of falling film dynamic crystallization.

### BRIEF DESCRIPTION OF THE DRAWINGS

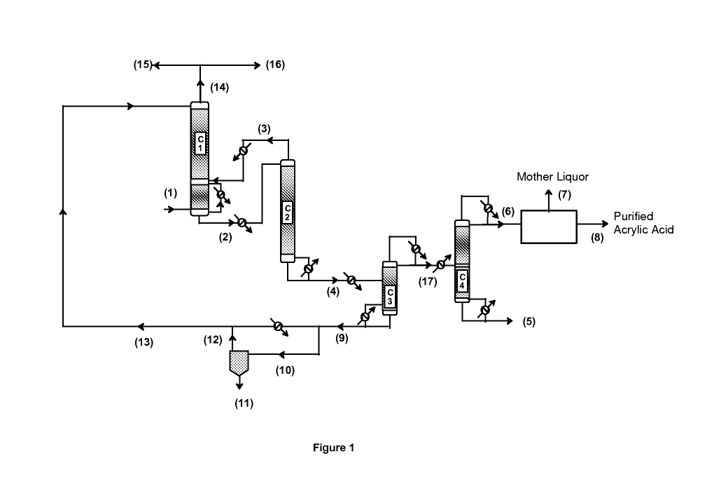

A better understanding of the process of the invention will be obtained on reading the description below, made with reference to FIGS. 1 to 4, which diagrammatically illustrate the various alternative embodiments. The symbols representing the main heat exchangers have been symbolized in the diagrams by a downward arrow for the cooling stages and an upward arrow for the heating stages.

### FIG. 1

This figure illustrates the preferred alternative form of the process of the invention. A gaseous reaction stream (1) is introduced at the bottom of the absorption column C1 which receives, countercurrentwise, a heavy hydrophobic solvent or a mixture of heavy hydrophobic solvents. At the column bottom, the liquid stream (2) still comprises water and light compounds (acetic acid in particular). It is sent to a distillation column C2, which makes it possible to recover the water and the light compounds (acetic acid) at the top, in the stream (3), which is recycled to the column C1. At the top of the column C1, the gas stream (14) comprises all the noncondensable compounds (nitrogen, oxygen, CO, CO₂) and light compounds (acetaldehyde, acrolein, acetic acid, water, and the like). This stream (14) can be partially recycled to the reaction (15) and partially or completely purged (16).

At the bottom of column C2, the liquid mixture comprises the AA (15-25%) in solution in the solvent, and the heavy intermediate compounds (with a boiling point between that of the AA and that of the solvent, such as maleic anhydride, furfural, benzaldehyde, protoanemonin, 2-butenoic acid, and the like) and the compounds optionally present which will be heavier than the solvent.

This stream (4) feeds the column C3 at the top. This column makes it possible to recover:
at the bottom, the solvent+traces of heavy compounds (9), with the minimum of acrylic acid,
at the top, a stream (17) which comprises most of the AA and of the intermediate compounds, and small concentrations of solvent (approximately 1%).

The stream (9) is recycled at the top of the absorption column C1, optionally after purging (10 and 11), in all or part, from the stream (9) the compounds heavier than the solvent in an evaporator, it being possible for the evaporator top stream comprising the solvent to be recovered (12).

The stream (17) is subsequently sent to a distillation column C4 which makes it possible to separate the AA of technical grade at the top (6) and the “heavy compounds” at the bottom (5), composed of the solvent and intermediates. This stream (5) can subsequently be purified in an additional column (not represented in the diagram) in order to remove the heavy intermediate compounds at the top and to recover, at the bottom, the solvent and the inhibitors, it being possible for the latter bottom stream subsequently to be recycled upstream of the process.

Due to the high acetic acid content of the reaction stream (1) produced ex-glycerol, the column C2 does not make it possible to remove all of this impurity. The stream of technical AA (6) still comprises acetic acid, along with propionic acid and 2-butenoic acid.

This stream (6) is purified by fractional crystallization, which makes it possible to simultaneously remove the acetic acid, the propionic acid and the 2-butenoic acid.

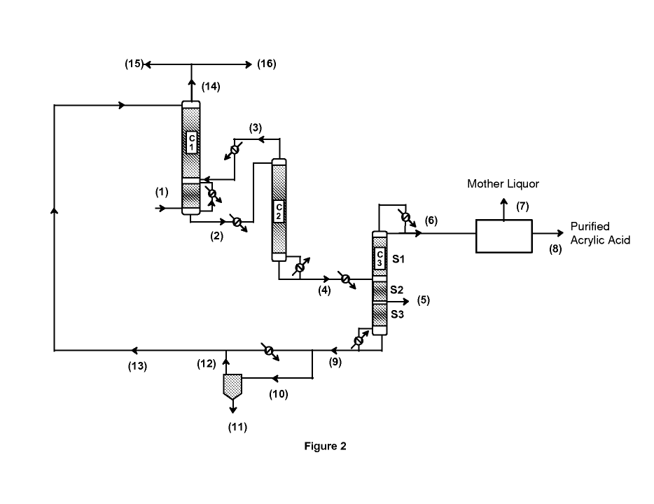

### FIG. 2

In this alternative form, the liquid phase resulting from stage (3) is subjected to
a topping by distillation with separation, at the top, of the water and residual light compounds (stage 4), the bottom fraction being sent to a stage (5),
a fractionation by distillation in a column fed at the level of an intermediate plate between the column bottom and top, equipped with a side stream withdrawal for the intermediate compounds, preferably in the gas phase and in a lower position than the feed level (situated between the feed plate and the column bottom), with withdrawal at the top for the acrylic acid and with withdrawal at the bottom for the solvent (stage 5),
a purification of the acrylic acid resulting from the top effluent stream from stage (5) by fractional crystallization.

In this alternative form, the stream (4) feeds a column C3 comprising 3 sections, from the top downwards S1, S2 and S3. This single column performs the functions of columns C3 and C4 of FIG. 1. Feeding by the stream 4 takes place at the bottom of the section S1. By side stream withdrawal from the column, between the section S2 and the section S3, a stream (5) is recovered which is rich in heavy intermediate impurities and which comprises a small amount of solvent and optionally of stabilizers. This stream can be treated as described above in order to recover the solvent and the stabilizer for the purpose of a recycling upstream of the process. At the bottom of the column, at the bottom of S3, the solvent and the heavy compounds are recovered in the stream 9 and are recycled to the absorption column after prior treatment via 10, 11, 12 and 13.

The stream (6) obtained at the top of column C3 is the technical AA, which can be purified by fractional crystallization.

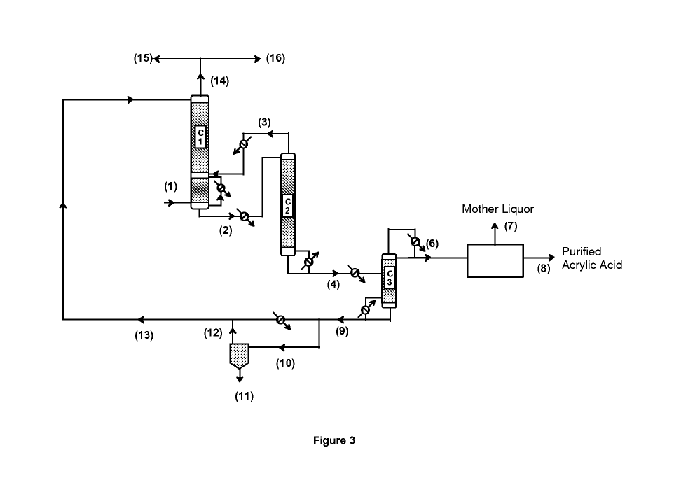

### FIG. 3

In this alternative form, the liquid phase resulting from stage (3) is subjected to
a topping by distillation with separation, at the top, of the water and the residual light compounds (stage 4), the bottom fraction being sent to a stage (5),
during which a fractionation by distillation is carried out, on the one hand, of the acrylic acid at the top and of the heavy solvent at the bottom (stage 5),
a purification of the acrylic acid resulting from the top effluent stream from stage (5) by fractional crystallization.

This figure illustrates a simplified alternative form of the process which makes it possible to dispense with the column C4 of FIG. 1. The stream 6 to be purified is richer in heavy impurities. In this case, it is no longer possible to recover the solvent entrained with the AA at the top of C3 as this solvent will be found in the mother liquors which comprise all the other impurities.

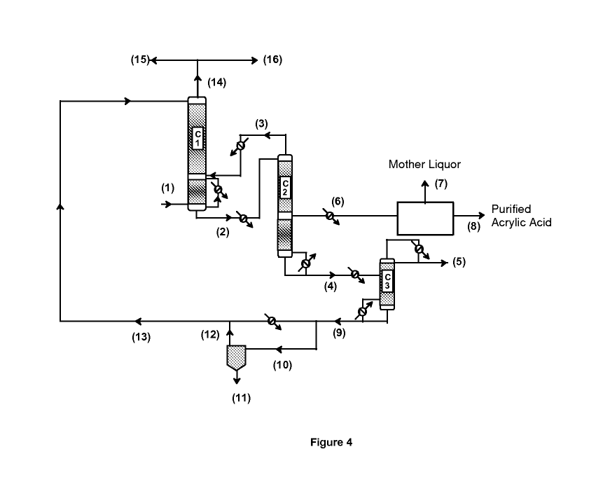

### FIG. 4

In this alternative form, the liquid phase resulting from stage (3) is subjected to
a fractionation by distillation in a region comprising two sections with separation at the top of the water and residual light compounds, at the bottom of the heavy solvent and, by side stream withdrawal, at the boundary of the two sections, of the acrylic acid (stage 4),
a purification of the acrylic acid resulting from the side stream withdrawal of stage (4) by fractional crystallization.

This is another alternative form where the acrylic acid is recovered by side stream withdrawal in the vapor phase in the column C2. This stream (6) is purified by crystallization. The bottom stream (4) of column C2 is sent to a column C3 for removal, to at the top, of a stream (5) comprising most of the heavy intermediate compounds, with a small amount of solvent. This stream (5) can subsequently be treated as described above in order to recover the solvent and optionally stabilizers, which will be recycled upstream of the process.

The invention also relates to the use of the bioresourced acrylic acid obtained according to the process of the invention in the manufacture of homopolymers and copolymers produced by polymerization of acrylic acid and optionally of other unsaturated monomers, for example the manufacture of superabsorbent polymers obtained by polymerization of said partially neutralized acid or the polymerization of said acid, followed by a partial neutralization of the polyacrylic acid obtained.

The invention also relates to the polymers and copolymers obtained by polymerization of bioresourced acrylic acid and optionally of other bioresourced monomers or monomers resulting from fossil starting materials.

The invention also relates to the superabsorbants obtained by polymerization of bioresourced acrylic acid.

The invention is also targeted at the use of bioresourced acrylic acid in the manufacture of polymers or copolymers by polymerization of the derivatives of said acid in the ester or amide form. It is also targeted at the polymers or copolymers obtained by polymerization of the derivatives, in the ester or amide form, of bioresourced acrylic acid.

The process of the invention is illustrated by the following examples.

### EXAMPLE 1

### Manufacture of Crude Acrylic Acid from Glycerol

The preliminary stage consists in purifying the crude glycerol obtained from vegetable oil, with removal of the salts. The crude glycerol solution is composed, by weight, of 89.7% of glycerol, 3.9% of water and 5.1% of sodium chloride. This stream (6400 g) is continuously conveyed as feed to a stirred 2-liter reactor heated by an external electrical reactor heater. The glycerol and water vapors are condensed in a reflux condenser and recovered in a receiver. This purification operation is carried out under a pressure of to 670 Pa (5 mmHg). 5710 g of a glycerol solution devoid of sodium chloride are obtained. Moving on to stage (1) of the process, the reaction for the dehydration of the glycerol to give acrolein and the condensation (1′) of a portion of the water are carried out. The dehydration reaction is carried out in the gas phase in a fixed bed reactor in the presence of a solid catalyst composed of a tungstated zirconia ZrO₂/WO₃ at a temperature of 320° C. at atmospheric pressure. A mixture of glycerol (20% by weight) and water (80% by weight) is conveyed to an evaporator in the presence of air in an O₂/glycerol molar ratio of 0.6/1. The gas medium exiting from the evaporator at 290° C. is introduced into the reactor, composed of a tube with a diameter of 30 mm charged with 390 ml of catalyst and immersed in a salt bath (KNO₃, NaNO₃ and NaNO₂ eutectic mixture) maintained at a temperature of 320° C.

At the outlet of the reactor, the gaseous reaction mixture is conveyed to the bottom of a condensation column. This column is composed of a lower section filled with Raschig rings surmounted by a condenser in which a cold heat-exchange fluid circulates. The cooling temperature in the exchangers is adjusted so as to obtain, at the column top, a temperature of the vapors of 72° C. at atmospheric pressure. Under these conditions, the loss of acrolein at the condensation column bottom is less than 5%.

In the following stage (2), the gas mixture is introduced, after addition of air (O₂/acrolein molar ratio of 0.8/1) and of nitrogen in an amount necessary in order to obtain an acrolein concentration of 6.5 mol %, as feed of the reactor for the oxidation of acrolein to give acrylic acid. This oxidation reactor is composed of a tube with a diameter of 30 mm charged with 480 ml of a commercial catalyst for the oxidation of acrolein to give acrylic acid based on mixed oxides of aluminum, molybdenum, silicon, vanadium and copper and immersed in a salt bath, identical to that described above, maintained at a temperature of 250° C. Before introduction over the catalytic bed, the gas mixture is preheated in a tube which is also immersed in the salt bath.

The description of the additional recovery and purification stages relates to the diagram of FIG. 1.

At the outlet of the oxidation reactor, the gas mixture (1) is introduced at the bottom of an absorption column C1, stage (3), operating at atmospheric pressure. This column is filled with random stainless steel packing of the ProPak type. In the lower part, over ⅓ of its total height, the column is equipped with a condensation section, at the top of which is recycled a portion of the condensed mixture recovered at the column bottom, after cooling to 70° C. in an external exchanger. A stream (14) composed of DTE (ditolyl ether), with a solvent/acrylic acid present in the reaction gas ratio by weight of 4/1, in which 0.5% of HQME has been dissolved beforehand as polymerization inhibitor, is fed at the column top at a temperature of 54° C. The temperature of the vapors at the column top is 52° C. and the temperature of the acrylic acid solution obtained at the column bottom is 84° C. The product (2) obtained at the bottom is cooled to a temperature of 35° C. and is then conveyed, using a pump, to the top of a column C2 equipped with 15 perforated plates having weirs. Distillation is carried out in this column at a pressure of 187 hPa. The temperature measured at the column bottom is 113° C. and the temperature of the column top is 88° C. All of the vapors condensed at the top (3) are returned in the external cooling loop of the column C1.

The stream (4) extracted at the foot of this column is crude acrylic acid, which assays 20.2% of acrylic acid. With respect to the acrylic acid, the concentration of impurities in the stream are 0.72% of acetic acid, 0.81% of propionic acid, 0.01% of furfural, 0.02% of protoanemonin, 0.03% of benzaldehyde, 0.04% of 2-butenoic acid and 0.41% of maleic anhydride.

The crude acrylic acid stream obtained in the preceding stage is sent as feed to a column C3 operating under a pressure of 117 hPa which is equipped with 4 perforated plates each provided with a weir and which is fed between the 2^{nd} and the 3^{rd} plates.

At the top of column C3, a portion of the condensed stream is returned at the level of the upper plate, with a reflux ratio (flow rate of liquid refluxed/flow rate of liquid withdrawn) of 0.2/1. The temperature measured in the reboiler is 180° C. and the temperature at the top reaches 119° C. The stream (9) obtained at the column bottom assays 0.082% of AA, i.e. a degree of recovery of the monomer at the column top of 99.7%.

The stream (17) of AA withdrawn at the top of column C3, comprising, as impurities, predominantly 0.67% of acetic acid, 0.78% of propionic acid, 0.01% of furfural, 0.02% of protoanemonin, 0.03% of benzaldehyde, 0.04% of 2-butenoic acid, 0.4% of maleic anhydride and 1.1% of ditolyl ether, is conveyed to the level of the 4^{th} plate (counting from the bottom) of a second column C4 equipped with 16 perforated plates provided with weirs. This column C4 operates under a pressure of 226 hPa (170 mmHg) and receives, at the top, a mixture of stabilizer (5% HQME in AA). The reflux ratio applied at the top (flow rate of liquid refluxed/flow rate of liquid withdrawn) is 1.5/1. The bottom temperature is 187° C. and the top temperature is 93° C. The technical acrylic acid obtained at the column top assays 98% of AA. The impurities present in this stream are acetic acid (0.68%), propionic acid (0.76%), furfural (0.005%), protoanemonin (0.009%), benzaldehyde (0.012%), 2-butenoic acid (0.016%), maleic anhydride (0.12%), water (0.21%) and DTE (0.005%).

### EXAMPLE 2

### Purification of the Ex-Glycerol Technical AA by Crystallization

The stream of acrylic acid of technical grade obtained in example 1 is subjected to a series of steps of purification and concentration by fractional crystallization, as described in the present patent application. The arrangement used is a falling stream crystallizer composed of a vertical stainless steel tube filled with heat-exchange fluid (ethylene glycol/water mixture) circulating in a closed circuit, via a pump, through an external heat exchanger which can be programmed as a temperature gradient (Lauda cryostatic bath). This tube is fed at the top in the form of a liquid film which flows uniformly over its external wall. The liquid composed of the mixture to be crystallized, recovered in a receiving tank at the bottom, recirculates as a loop in a lagged circuit in order to again feed the tube at the top, via a pump.

The stream of technical acrylic acid is subjected to a series of several successive purification steps, each step comprising the following stages:
crystallization: the heat-exchange fluid is rapidly cooled, so as to lower the temperature of the falling film of acrylic acid down to the temperature of crystallization of the acrylic acid in the mixture, determined beforehand from a sample of the mixture to be purified, and then a negative temperature gradient, of 0.1 to 0.5° C./min, is imposed on the heat-exchange fluid. When the volume of crystallized acrylic acid, measured by difference by evaluating the level of liquid in the collecting container at the bottom of the crystallizer, reaches 70% of the starting mixture, the recirculation of the falling film of mixture to be purified is halted and the tube is drained. The liquid mixture of the mother liquors thus obtained is separated and stored in a receiver.
sweating: the heat-exchange fluid is reheated, so as to bring about the melting of a portion (5%) of the layer of crystallized acrylic acid at the surface of the tube. The mother liquors from this sweating stage are collected and stored in the same receiver as the mother liquors from the preceding stage.
melting: the heat-exchange fluid is rapidly reheated up to a temperature of 30° C., until the crystallized layer has completely melted. The purified liquid stream is placed in a different receiver.

The product purified by melting in the final stage of the first purification step is conveyed to the second purification step, where it will be subjected to a new series of the 3 purification stages under the same operating conditions. The mother liquors from the second purification step are subsequently mixed with a fresh charge of the feed stream of technical AA in step 1. This process is thus repeated until the desired grade is obtained in the molten purified product.

In order to limit the losses of acrylic acid which are concentrated in the mother liquors from the first purification step, a series of successive concentration steps, exhibiting the same stages as the purification steps, is carried out in which the crystallisate from the “n−1” step is conveyed as feed of the “n” step and the mother liquors from this “n−1” step are conveyed as feed of the “n−2” step. These steps are carried out under the same operating conditions as the purification steps, except for the volume of crystallized acrylic acid targeted, before passing from the crystallization stage to the sweating stage, which is 60% of the product fed.

The final crystallization step is carried out in static mode. The stream to be purified is placed in a container made of stainless steel with a jacket through which circulates a cooled fluid maintained at the crystallization temperature of the medium, determined beforehand by a measurement of crystallization temperature. A vertical tube made of stainless steel filled with heat-exchange fluid (ethylene glycol/water mixture) circulating in a closed circuit, via a pump, through an external heat exchanger which can be programmed as a temperature gradient is immersed in this container.

In a first stage, the temperature of the heat-exchange fluid in the tube is rapidly lowered to the crystallization temperature of the medium and then a negative temperature to gradient of 0.1 to 0.5° C./mn is imposed. When the crystallized volume reaches approximately 50% of the starting material, the mother liquors are removed, a sweating stage is then carried out and, finally, the melting stage is carried out, as in the upper crystallization steps in dynamic mode.

Applied to the technical acrylic acid obtained from glycerol on completion of the purification stages of example 1, a sequence of 4 purification steps and 3 concentration steps made it possible to obtain acrylic acid of “glacial” grade comprising 50 ppm of acetic acid, 410 ppm of propionic acid, less than 1 ppm of maleic anhydride, less than 80 ppm of water, less than 1 ppm of 2-butenoic acid, less than 1 ppm of furfural, less than 1 ppm of benzaldehyde, less than 1 ppm of protoanemonine and less than 1 ppm of acrolein.

The concentration of acrylic acid in the residual mother liquors from the final concentration step is 71%. The AA recovery yield in this purification stage is 97.2%. With an additional concentration step in static mode, the AA concentration in the final mother liquors is 54.3% and the overall purification yield is 99.3%. The residue has the following composition by weight: AA: 54.3%; water: 7.3%; maleic anhydride: 8.9%; protoanemonin: 1%; benzaldehyde: 2%; acetic acid: 4.3%; propionic acid: 16.7%; acrolein: 1.6%; furfural: 0.8%; 2-butenoic acid: 2%.

The acrylic acid produced according to the invention is a bioresourced acid manufactured from nonfossil natural starting materials.

The use of nonfossil carbon-based starting materials of natural origin can be detected by virtue of the carbon atoms participating in the composition of the final product. This is because, unlike fossil substances, substances composed of renewable starting materials comprise the radioactive isotope ¹⁴C. All carbon samples drawn from living organisms (animals or plants) are in fact a mixture of 3 isotopes: ¹²C (representing ˜98.892%), ¹³C (˜1.108%) and ¹⁴C (traces: 1.2×10⁻¹⁰%). The ¹⁴C/¹²C ratio of living tissues is identical to that of the CO₂ of the atmosphere.

The invariableness of the ¹⁴C/¹²C ratio in a living organism is related to its metabolism, with continual exchange with the atmosphere.

The disintegration constant of ¹⁴C is such that the ¹⁴C content is virtually constant from the harvesting of the plant starting materials up to the manufacture of the final product.

The bioresourced acrylic acid obtained by the process of the invention has a content by weight of ¹⁴C such that the ¹⁴C/¹²C ratio is greater than 0.8×10⁻¹² and preferably greater than 1×10⁻¹².

The measurement of the ¹⁴C content of substances is clearly described in the standards ASTM D6866 (in particular D6866-06) and in the standards ASTM D7026 (in particular 7026-04).

1. A process for the manufacture of bioresourced acrylic acid from glycerol, comprising the following stages:
gas-phase catalytic dehydration of glycerol to give acrolein, (1)
partial condensation by cooling and extraction of a portion of the water and heavy compounds present in the reaction medium of (1), (1′)
gas-phase catalytic oxidation of the acrolein to give acrylic acid, (2)
extraction of the acrylic acid present in the effluent stream from the oxidation by countercurrentwise absorption with a heavy hydrophobic solvent with cooling and removal, at the top, of the light fraction composed of the “noncondensable” gaseous compounds and condensable light compounds, such as water, acetaldehyde, unconverted acrolein, formic acid or acetic acid, (3)
separation of the residual light fraction and the heavy solvent present in the liquid phase resulting from stage (3) by at least one distillation stage (4), (5) and/or (6) and recovery of the acrylic acid fraction thus separated, and
purification of the acrylic acid present in the acrylic acid fraction resulting from the preceding stage(s) by fractional crystallization.

2. The process as claimed in claim 1, characterized in that the liquid phase resulting from stage (3) is subjected to
a topping by distillation with separation, at the top, of water and residual light compounds (stage 4), the bottom fraction being sent to stage (5),
a distillation of the acrylic acid solution thus obtained in order to separate, at the bottom, the heavy solvent and, at the top, the acrylic acid fraction comprising the intermediate impurities (stage 5),
a distillation of the acrylic acid solution resulting from the preceding stage (5) in order to remove, at the bottom, the heaviest “intermediate” compounds and, at the top, the acrylic acid (stage 6),
a purification of the acrylic acid resulting from stage (6) by fractional crystallization.

3. The process as claimed in claim 1, characterized in that the liquid phase resulting from stage (3) is subjected to
a topping by distillation with separation, at the top, of the water and residual light compounds (stage 4), the bottom fraction being sent to a stage (5),
a fractionation by distillation in a column fed at the level of an intermediate plate between the column bottom and top, equipped with a side stream withdrawal for the intermediate compounds, preferably in the gas phase and in a lower position than the feed level (situated between the feed plate and the column bottom), with withdrawal at the top for the acrylic acid and with withdrawal at the bottom for the solvent (stage 5),
a purification of the acrylic acid resulting from the top effluent stream from stage (5) by fractional crystallization.

4. The process as claimed in claim 1, characterized in that the liquid phase resulting from stage (3) is subjected to
a topping by distillation with separation, at the top, of the water and the residual light compounds (stage 4), the bottom fraction being sent to a stage (5),
a fractionation by distillation, on the one hand, of the acrylic acid at the top and of the heavy solvent at the bottom (stage 5),
a purification of the acrylic acid resulting from the top effluent stream from stage (5) by fractional crystallization.

5. The process as claimed in claim 1, characterized in that the liquid phase resulting from stage (3) is subjected to
a fractionation by distillation in a region comprising two sections with separation at the top of the water and residual light compounds, at the bottom of the heavy solvent and, by side stream withdrawal, at the boundary of the two sections, of the acrylic acid (stage 4),
a purification of the acrylic acid resulting from the side stream withdrawal of stage (4) by fractional crystallization.

6. The process as claimed in claim 1, characterized in that the fractional crystallization stage is carried out according to the falling film fractional crystallization technique.

7. The process as claimed in claim 6, characterized in that the fractional crystallization stage comprises at least 2 purification steps and between 1 and 4 steps for the concentration of the impurities.

8. The process as claimed in claim 6, characterized in that the fractional crystallization stage is supplemented by a step of concentration by static crystallization.

9. The process as claimed in claim 1, characterized in that the heavy hydrophobic solvent is chosen from aromatic solvents exhibiting a boiling point of greater than 260° C. and comprising one or two aromatic ring systems substituted by at least one alkyl radical having from 1 to 4 carbon atoms or one cycloalkyl radical.

10. The process as claimed in claim 9, characterized in that the solvent is ditolyl ether, alone or in the form of a mixture of its isomers, or the ditolyl ether and dimethyl phthalate mixture.
